(19) United States
(12) Patent Application Publication
(10) Pub. No.: US 2005/0019380 A1
(43) Pub. Date: Jan. 27, 2005

(54) Microbial cellulose wound dressing for treating chronic wounds

(75) Inventors: Russell Hoon, Doylestown, PA (US); Gerry Ann Oster, Langhorne, PA (US); Chris Damien, Newtown, PA (US); Jonas Chia-Tsung Wang, West Winder, NJ (US); Gonzalo Serafica, Langhorne, PA (US)

Correspondence Address: FOLEY AND LARDNER;SUITE 500, 3000 K STREET NW, WASHINGTON, DC, 20007, US

(73) Assignee: Xylos Corporation

(21) Appl. No.: 10/864,804

(22) Filed: Jun. 10, 2004

Related U.S. Application Data
(63) Continuation-in-part of application No. 10/732,802, filed on Dec. 11, 2003.
Application No. 10/732,802 is a continuation-in-part of application No. 10/132,171, filed on Apr. 26, 2002.

(51) Int. Cl. A61F 15/00; A61L 15/00

(52) U.S. Cl. 424/445; 602/1

(57) ABSTRACT

The invention relates to a dressing comprised of microbial-derived cellulose for aesthetic application The dressing is capable of donating liquid to dry substrates and is also capable of absorbing exudating wounds. Delivery of various medicaments using the dressing is possible. Positive clinical outcomes including reduced pain and discomfort, faster epithelialization, and healing were observed with use of the dressing.

### CROSS-REFERENCE TO RELATED PATENT APPLICATIONS

This application is a Continuation in part of U.S. application Ser. No. 10/732,802, filed Dec. 11, 2003 (pending), incorporated herein by reference in its entirety, which is a Continuation in part of U.S. application Ser. No. 10/132,171, filed Apr. 26, 2002 (pending), incorporated herein by reference in its entirety.

### FIELD OF THE INVENTION

The invention relates to a cosmetic dressing comprising a microbial-derived cellulose for use on facial skin and for other aesthetic applications. The dressing can be used for treatment of the facial skin or other epidermal/dermal tissue after procedures including microdermabrasion, chemical peels, non-ablative as well as ablative laser resurfacing, and other treatments of the skin. The form of the dressing is that of a facial mask, shape variations thereof, or additional shapes to fit other epidermal/dermal treatment areas. The invention also relates to a microbial-derived cellulose dressing, in the shape of a facial mask or shapes suitable for other treatment areas, containing medicaments or agents for optimizing the healing of facial infirmity. The invention further relates to an improved method for treating facial skin with a facial mask or treating other areas of the body with a dressing, with or without medicaments or agents for optimizing the healing, reduced pain and discomfort, for treating skin disorders, anti-wrinkle, depigmentation, and producing the desired clinical outcomes such as faster healing and more aesthetically pleasing result.

### BACKGROUND OF THE INVENTION

There are a wide variety of materials used to fabricate wound dressings, which are used to treat a host of surgical and non-surgical lesions, such as burns and abrasions. The dressings range from simple gauze-type dressings to animal derived protein-type dressings such as collagen dressings, the composition of the particular dressing depends on the type of wound to be treated. Each of these dressings has advantages depending upon the type of application. For example, gauze-type dressings are sufficient and highly economical for simple abrasions and surgical incisions.

However, for epidermal/dermal tissue wound and aesthetic application, no current product delivers the changing requirements of the area being treated. For example, with laser ablated skin, the optimal dressing requires specific properties depending on the stage of healing. Specifically, the ideal dressing should be able to 1) establish hemostasis and absorb exudate during inflammatory phase, 2) provide the ideal environment to help promote epithelial migration in the proliferative phase and lastly, 3) support maturation and remodeling of the epidermis. (Skover, G. et al.).

To achieve such product requirements, various types of polymeric materials have been used in the treatment of facial skin disorders. Generally, they can be broken down into two major classes, namely synthetic and naturally derived polymeric materials.

Synthetic materials include silicone, polyurethanes, polyvinylpyrolidone (PVP), polyethylene oxide (PEO), polyvinyl alcohol (PVA), and polyacrylonitrile (PAN). These materials may be used in combination with other synthetic or natural polymers to fabricate wound dressings with specific properties such as moisture retention and high fluid absorption. Both of these properties, generally not found in gauze-type dressings, promote healing by protecting wounds from infection and maintaining moisture levels in the wound. Huang discloses in U.S. Pat. No. 6,238,691 a three dimensional cross-linked polyurethane hydrogel wound dressing, which is absorptive, contours to a wound site and maintains the wound in a moist state to promote healing. Silicone and other synthetic polymer-based products have also been applied for treating laser resurfaced facial skin (Newman, J. P. et al., 1998). These investigators have shown that these dressings improve patient comfort and simplify their post operative wound care without the increased risk of infection.

Similarly, naturally derived polymers or biopolymers, such as collagen and alginates, have also been used as wound dressings that exploit the desirable characteristics of the polymers, such as high absorption capacity of alginate or the biocompatible nature of collagen. Recent investigators (Goldman et al., 2002) have shown that the use of collagen dressings may help in epithelial regeneration better than silicone based materials. Each of these dressings has associated particular advantages depending on the type of wound and amount of exudate it generates. However, these dressings also have disadvantages, which include higher cost, wound adherence, limited exudate absorption and residue deposition on a wound site.

As an alternate material, microbial-derived cellulose possesses inherent characteristics which allow for effective promotion of wound healing without some of the inherent disadvantages associated with current wound dressings. In this regard, microbial-derived cellulose possesses physical properties that distinguish it from plant-derived cellulose such as extreme hydrophilicity and unique multi-layered three dimensional laminar structures that provide its moisture handling ability. Microbial cellulose is highly hydrophilic with a water-holding capacity ranging from 60 to 700 times its own weight as is described in U.S. Pat. No. 4,942,128. Microbial cellulose also demonstrates excellent wet strength and does not breakdown under compression. Lastly, because of its laminar multi-layered structure, microbial cellulose can be processed to produce a film with novel fluid handling ability. By adjusting the cellulose to liquid ratio, processed microbial cellulose is capable of both donating fluid and absorbing liquid depending on the surface with which the film is made to come in contact.

Because of its superior characteristics, use of microbial cellulose in the medical industry has been previously investigated. For example, U.S. Pat. Nos. 4,588,400, 4,655,758 and 4,788,146 to Ring et al. disclose the possible use of microbial-derived cellulose in liquid-loaded medical pads. The patents to Ring et al. focus on using statically produced microbial cellulose pads loaded with various liquids and medicaments. Various types of liquids that can be contained in the microbial cellulose pad were detailed as well as the production and cleaning method to produce the starting cellulose material. Also described in these patents are examples which detail methods of fabrication of various pads wherein the method involves a series of pressing and soaking steps to adjust the physical properties, mainly with respect to the liquid to cellulose ratio to yield a desired product. As an example, these patents illustrate a highly hydrated pad (80 to 1 fluid to cellulose ratio) that is able to provide a cooling capability which is ideal for burn applications. In particular, the '146 patent describes the use of such liquid loaded pads as wet dressings for use as an ulcer dressing capable of providing moisture to the wound over an extended period of time. The same '146 patent also mentions that the wet dressings described in the examples have the additional ability to absorb large quantities of fluid from the wound site when the dressing is applied in a less than saturated condition. However, the wound dressings of Ring et al. fail to mention a singular dressing having both the ability to be a source of moisture as well as the ability to absorb fluid. The Ring et al. patents also fail to describe the effective liquid to cellulose ratio to fabricate a dressing having the dual fluid handing capability and also fail to disclose any particular benefits from the combination of microbial cellulose and ascorbic acid ( Vitamin C) and/or its salts or esters, retinol (Vitamin A) and /or its esters, alpha tocopherol (Vitamin E) and/or its esters, alpha hydroxyl acid, oleic acid, MMP inhibitors and a buffer solution to maintain the dressing at a pH range of 4 to 8. Furthermore the Ring et al. patents do not disclose the use of microbial-derived cellulose for the treatment of subacute/acute facial wounds, including wounds generated by microdermabrasion, chemical peels, and non-ablative as well as ablative laser resurfacing procedures, or that the form of the dressing is that of a facial mask.

U.S. Pat. No. 4,912,049 to Farah et al. discloses the use of statically produced dehydrated microbial cellulose as an artificial skin graft, a separating membrane or artificial leather. The '049 patent recites the use of a cellulose film formed by Acetobacter xylinum that is dehydrated while it is stretched. Although the '049 patent described potential use of their invention as an artificial skin for treatment of wounds or injury, there is no suggestion that the material could be used for facial skin and aesthetic applications. Furthermore, the dried film of Farah has no moisture donation capability and minimal absorption capacity. Farah fails to disclose any particular benefit from the combination of microbial cellulose and medicaments or agents that can help promote healthy skin.

U.S. Pat. No. 5,846,213 by Wan et al. discloses methods of preparing microbial cellulose films using raw material produced in a stirred-tank bioreactor, instead of the static method. The '213 patent further describes the use of such cellulose material dissolved in solvents to fabricate membranes that can be use as wound dressings. Because of its dry nature of the resulting film, the cast material lacks any moisture donating ability and limited fluid absorption capacity. Also, the resulting cellulose membrane does not possess the three dimensional multi-layered structure found only in statically grown microbial cellulose as previously described. As with Ring et al. and Farah, no disclosure of any particular benefit from the combination of microbial cellulose and medicaments or agents that can help in promoting healthy skin.

Although the above patents recognize the potential use of microbial cellulose in medical applications, the prior art has failed to provide a method of developing a wound dressing that demonstrates optimal wound healing, moisture management capability, the ability to enhance wound healing, pain reduction and adequate biocompatibility. Accordingly, an effective wound dressing comprising microbial cellulose for treatment of chronic wounds, which is highly biocompatible, is desirable. Furthermore, a wound dressing with high moisture donation and absorption capabilities is also particularly desirable for optimal wound healing. This dual moisture handling ability of the dressing of the present invention is capable of maintaining a moist wound environment necessary for healing wounds. Additionally, the ability of the wound dressing, of the present invention, in assisting autologous healing by promoting granulation and allowing epithelial cells to migrate, endows the distinct ability of the wound dressing to effect wound closure and enhance the aesthetic appearance of the skin. Finally, the incorporation of medicaments into the wound dressing will further enhance the promotion of healthy skin and improve the skin's aesthetic appearance.

Thus, the present inventors have developed an epidermal/dermal tissue dressing which possesses this novel fluid handling capability of absorption and donation. This fluid handling capability is an end result of the processing microbial cellulose to contain the proper cellulose content for the intended purpose. The resulting dressing can donate fluid if the surface of the tissue site is dry. The same dressing is also capable of absorbing fluid away from exuding tissue. Additionally, the microbial cellulose dressing described in this invention will not degrade and leave a residue in the treatment area site. Removal of the microbial cellulose dressing from the treatment area does not damage tissue because it does not adhere to the tissue surface.

The present invention also envisages microbial cellulose sheets which can be directly synthesized in virtually any shape or size and for any area comprising epidermal/dermal tissue, although for particular aesthetic applications, a facial shape is desirable. Fermentation processes yield an extremely thin and pliable form, which is remarkably strong yet gas and liquid permeable. The shape will remain intact even when subjected to extreme environmental conditions such as autoclaving or gamma sterilization.

### SUMMARY OF THE INVENTION

It is an object of the present invention to provide methods for aesthetically treating epidermal/dermal tissue with a microbial-derived cellulose dressings 1.5 to 9% cellulose by weight. In a preferred embodiment, the microbial-derived cellulose is biocompatible and nonpyrogenic.

It is another object of the present invention to provide an effective aesthetic dressing comprising microbial cellulose, for treatment of facial skin or other epidermal/dermal tissue, that is capable of donating and absorbing moisture for optimal tissue healing or improvement in the form of a facial mask or suitably shaped dressing.

It is a further object of the present invention to enhance the tissue healing process by incorporation of medicaments including ascorbic acid (Vitamin C) and/or its salts or esters, retinol (Vitamin A) and /or its esters, alpha tocopherol ( Vitamin E) and/or its esters, alpha hydroxyl acid, oleic acid, MMP inhibitors, growth factors (e.g. epidermal growth factor (EGF), etc.), antimicrobial or antiseptic agents (e.g. polyhexamethylene biguanide (PHMB), etc.) and a buffer solution to maintain the dressing at a pH range of 4 to 8 depending on what is desired.

It is a final object of the present invention to provide an improved method for treating facial skin, with a facial mask, or other sites of epidermal/dermal tissue, with appropriately shaped dressing, with or without an agent for optimizing the healing of facial skin, reducing discomfort and improving the tissue's aesthetic appearance.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1: The absorption and donation capabilities of microbial cellulose dressings are shown versus the percent cellulose contained in the materials. All materials were of identical area and similar thickness. The region of intersection of the two curves shows the ideal cellulose content to maximize both properties.

FIG. 2: The amount of fluid donated to a dry surface from XCell microbial cellulose dressing and from hydrogel wound dressings is shown. Donation quantities are expressed as a percent of the original sample weight. The donation of the XCell wound dressing is markedly superior to that of the hydrogels.

FIG. 3: The absorption and donation capabilities of XCell microbial cellulose dressing are compared to that of Clearsite (NDM) hydrogel wound dressing. The absorptive capacity is nearly identical for the two, but the XCell wound dressing can donate 6 times more than the hydrogel.

FIG. 4: The donation capability of microbial cellulous dressings is decreased over time when Telfa is placed between the leather and XCell.

### DETAILED DESCRIPTION OF THE PREFERRED EMBODIMENTS

The invention provides methods for treating facial skin and other epidermal/dermal tissue areas with microbial-derived cellulose. The invention also provides biocompatible, nonpyrogenic microbial-derived dressings with proper cellulose to liquid ratio as well as, liquid donation and absorption capability for optimal tissue healing and/or for aesthetic improvement. Unlike hydrocolloid, hydrogel, alginate, collagen or gauze dressings, the microbial-derived cellulose dressing described herein can provide an optimal moist healing environment by donating fluid to dry surface or absorbing excess fluid from exudating tissue.

The content of microbial-derived cellulose present in the dressing can fluctuate depending upon the method of preparation and the eventual end use of the dressing. In the present invention, the amount of microbial-derived cellulose present in the dressing is about 1.5% to about 9%; preferably it is about 3% to about 7%, more preferably about 4% to about 6% by weight.

The dressing of the present invention can be used for moisture donation. Typically, the dressing can donate up to 100% of its liquid weight to a dry substrate. This means that epidermal/dermal tissue which exhibits dry necrotic tissue can be effectively treated by application of a fluid containing dressing.

The dressing of the present invention also can be used for moisture absorption. Typically, the dressing can absorb up to about 200% of its weight. This means that epidermal/dermal tissue that is exudating can be effectively treated by application of a dressing of the present invention which will absorb excess fluid from the tissue. The exudation stage usually occurs when the dermis begins to form granulation tissue to fill up the space the dead dermal tissue use to occupy. At this stage the dressing of the present invention is able to absorb the fluid exudate while keeping a moist surface for epithelial cells to migrate. The epithelial migration is essential for eventually closing the wound. Thus, the dressing of this invention is able to provide optimum conditions for epidermal/dermal tissue healing and/or for aesthetically improving the treated tissue's appearance due to its dual ability to absorb and donate moisture.

1. Production of Microbial Cellulose under Static Conditions for Testing Procedures

In preparing the microbial cellulose of the invention, microorganisms such as Acetobacter xylinum are cultured in a bioreactor containing a liquid nutrient medium at 30 degrees and at an initial pH of 3-6. The medium is based on sucrose or other carbohydrates. Preferably, efficient film production is achieved using sucrose as a carbon source, ammonium salts as a nitrogen source, and corn steep liquor as nutrient source coupled with a proprietary trace elements supplement, which varies from the original Schramm & Hestrin medium (1954) used by those skilled in the art.

Suitable bioreactors are selected which minimize evaporation and provide adequate oxygen-limiting conditions. Oxygen-limiting conditions may be varied depending upon the desired water content and thickness of the cellulose film. Generally, under oxygen-limited conditions, oxygen is present in an amount of 5%-21% of the total gas present at the air liquid interface. The bioreactor is composed of plastic box fitted with an airtight cover or a limited gas-permeable cover. Dimensions of the bioreactor can vary in configuration depending on the shape and size of the cellulose film being produced. By limiting the amount of oxygen in the fermentation medium, it is hypothesized that the Acetobacter utilizes the carbon available in the medium to produce more cellulose instead of using it for reproduction, thereby increasing the total yield of cellulose.

The fermentation process under static conditions is allowed to progress over a period of about 7-30 days, during which the bacteria in the culture medium produce an intact cellulose pellicle containing the microorganisms. Depending on the desired thickness, which corresponds to certain cellulose content per unit area, the fermentation is stopped and the pellicle is removed from the bioreactor. The excess medium contained in the pellicle is then removed by standard separation techniques such as compression or centrifugation prior to chemical cleaning and subsequent processing of the pellicle to yield a dressing with cellulose to liquid ratio of about 1:10 to about 1:40. The raw cellulose pellicle has an increased sugar: cellulose yield of about 35%, compared to literature values of 10%.

2. Processing and Depyrogenation Procedures

Following production of the cellulose film, the cells have to be removed from the cellulose pellicle for purification. Fontana et al. (1990, Appl. Biochem. Biotech, 24: 253-264) have described the cells as being apyrogenic, however, the unpurified cellulose pellicle has tested positive for pyrogens using the Limulus Amebocyte Lysate (LAL) test kit. This result necessitated the removal of the cells by chemical processing discussed here in order to pass the standard pyrogenicity test and qualify the microbial cellulose dressing as nonpyrogenic.

The cellulose pellicle is subjected to a series of chemical wash steps to convert the raw cellulose film into a medical grade and non-pyrogenic dressing material. Typical processing uses hydroxide solutions at concentrations of 1-20% by weight. Preferably, sodium hydroxide is used at a concentration of not less than 2% and most preferably about 3% to about 5% in order to acetylate and eventually dissolve the cells. In addition, the present invention provides hydrogen peroxide washing capable of bleaching and sterilizing the pyrogen-free films. Concentrations of about 0.05% to about 10% peroxide by weight are useful to effect whitening of the films. Preferably the amount of peroxide used in about 0.1% to about 0.5%.

Purification processes using various exposure times, concentrations and temperatures were conducted on the raw fermentation product. Processing times of 1-4 hours have been studied in conjunction with temperature variations of 30-100 degrees centigrade to optimize the process. The resulting films from each of the different operating conditions were tested for their respective pyrogen levels and physical characteristics. The process condition that yields a nonpyrogenic product in the least amount of time and lowest chemical concentration was then selected for economic reasons. The time involved in this process can be as much as 4 hours at about 90° C.; preferably the time involved is about 1-2 hours at about 60° to about 80° C.

The amount of pyrogenic material left in the cellulose pad after processing may be measured by Limulus Amebocyte Lysate (LAL) test as outlined by the U.S. Food and Drug Administration (FDA) in 21 CFR10.90. The instant cleaning process outlined above provided a nonpyrogenic cellulose pad (<0.5 EU/ml). The allowable pyrogen content in Class I medical devices is 0.5 EU/ml (FDA LAL test Guideline). The steps of the LAL test are defined by the test kit manufacturer and can simply be followed to yield the pyrogen level in the cellulose film.

3. Physical Modification of Microbial Cellulose Dressing

Desirable characteristics of a microbial-derived cellulose dressing include an ability to provide a moist environment, and yet at the same time, the ability to absorb excess exudate fluid from, or donate moisture to, a treatment area. Currently marketed hydrogel wound dressing products have an approximate composition of 90-95% water and 5-10% polymer material. However, these products fail to provide adequate moisture to the wound and are characterized by inadequate strength. Furthermore, these dressing tend to adhere to the skin or wound site. This wound adhesion results in reinjury of the wound upon removal. The dressings of the instant invention however display superior moistness and absorptivity due to a laminar multi-layered three-dimensional structure not found in any other wound dressing. The cellulose dressing has also displayed the ability to control the level of moisture in the dressing treatment area interface by absorbing excess fluid or donating moisture depending on the conditions at the tissue treatment site. This moisture management capability helps in the promotion of epidermal migration leading to healing, as well as reduction of pain and discomfort and is a novel characteristic of the cellulose dressing.

4. Product Packaging and Sterilization

Packaging material should be impermeable to water to prevent the moist cellulose dressing from drying out, and be able to withstand the sterilization process. For example, an aluminum plastic-coated heat-sealable chevron pouch provides adequate impermeability and moisture retention.

The two most commonly used sterilization procedures for medical wound dressings, gamma irradiation and electron beam sterilization, were both investigated. The packaged cellulose dressings were exposed at different levels of radiation ranging from 5-50 KGy. The sterility of each dressing was then evaluated using standard USP sterility tests. The overall appearance and mechanical integrity of the dressing and the packaging material were also examined. The results of the sterility testing showed that the cellulose dressing was stable at the 5-40 KGy radiation dose and a minimum dose of 15 KGray was required to assure product sterility. Cellulose dressing products that were to be used for the biocompatibility, animal and human tests were then all sterilized at about 30 KGy (two-fold safety factor) to assure product sterility.

5. Fabrication of Facial Masks

Prior to packaging and sterilization, the cleaned cellulose pellicle can be cut to specific shapes. For example, a 9×12 cellulose sheet can be cut into full facial shapes and smaller patches. One possible design is to divide the face into an upper (forehead to the nose level) and lower section (just below the nose to the chin/jaw area). Possible dimension using an average values for male and female are 10.5″ W×6.25″ H for the upper portion and 10.75″ W×3.25″ H for the lower portion. Therefore, dressing's dimensions cover a range of 8″ to 12″ width by 4″ to 7″ height for the upper portion and a range of 8″ to 12″ width by 2″ to 6″ height for the lower portion. Two eyeholes can be used for the upper portion and a mouth opening is provided for the lower portion. The cellulose dressing is non-adherent; thus, would need to be secured in place with a protective goggle, or using adhesive tape or a secondary covering with adhesive capability as detailed below.

A secondary covering may be affixed on one side of the cellulose sheet to further enhance its effectiveness and ease of use. The use of polyurethane films or impregnated knitted gauze that can regulate moisture vapor transmission can be used for this purpose. The attachment can be done by dehydrating certain areas of the cellulose dressing and using these dehydrated areas as contact points for the secondary covering. The secondary covering can also be used to help in holding the dressing assembly in place during use by having some of the secondary material extend beyond the perimeter of cellulose dressing and adhere partially to the skin. Alternatively the dressing can be “sandwiched” between two layers of material that are affixed to each other (like a tea bag configuration). One side can be porous such as Telfa® material to allow moisture transmission.

6. Addition of Medicaments

In order to enhance the effectiveness of the microbial cellulose dressing, incorporation of medicaments can performed prior to cutting, packaging and sterilization. Active agents that are beneficial for aesthetic application may be added to the dressing at desirable concentrations. Such agents can include but are not limited to ascorbic acid (Vitamin C) and/or its salts or esters, retinol (Vitamin A/retinoic acid) and/or its esters, alpha tocopherol (Vitamin E) and/or its esters, alpha hydroxy acid and family (lactic acid, gluconic acid, B-hydroxy acid), oleic acid, MMP inhibitors, growth factors (e.g. epithelial growth factor (EGF), Bone Morphogenetic Protein (BMP), etc.), antimicrobial or antiseptic agents (e.g. polyhexamethylene biguanide (PHMB), etc.) and a buffer solution to maintain the dressing at a pH range of 4 to 8 depending on what is desired.

### EXAMPLES

### Example 1

### Absorption/Donation Studies

Cellulose pellicles of varying thickness were produced and processed to remove cellular debris. Pellicles were compressed to a uniform thickness of 1.9 mm, yielding a series of films with cellulose contents ranging from 1.5% to 10%. These films were tested for the ability to absorb saline from a saturated surface, and to donate moisture to a dry surface.

Weighed samples of uniform area were placed on the surface of a saturated sponge. Saline was poured around the sponge to maintain saturation. After 24 hr, the samples were reweighed to determine absorption, which was then plotted as percent of initial sample weight. To determine the moisture donation, weighed samples of uniform area were placed on the surface of smooth, dry leather. The leather was weighed prior to addition of sample. After 2 hr, the sample was removed and the leather was reweighed to determine the quantity of moisture that was donated, which again was plotted as percent of the initial sample weight.

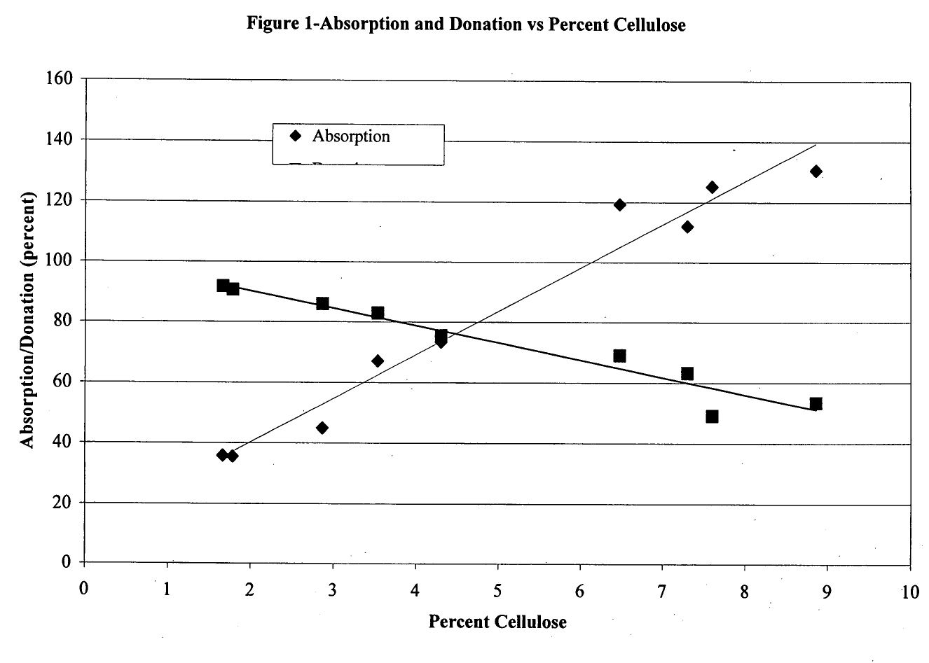

Both absorption and donation data were plotted on one graph to determine the optimal water content for both properties. This data is shown in FIG. 1. From this figure it can be seen that in order to possess absorption and donation capability, the cellulose percentage range in the dressing should be 1.5% to 9%, preferably 3% to 7%, more preferably 4% to 6% and most preferable 3% to 6%. The figure also shows that one could make a dressing that would have either enhanced absorption or enhanced donation, at the expense of the other property.

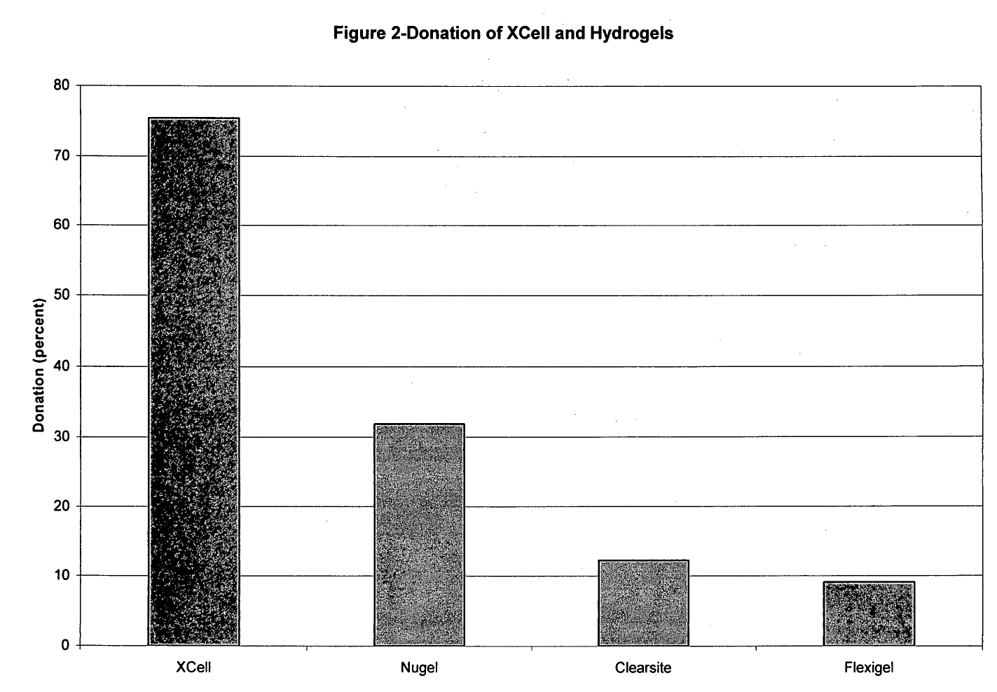

In order to show the superiority of the donation capability of the microbial cellulose dressing (XCell), tests were performed on traditional hydrogels in the market. Products tested were Clearsite (NDM), Nugel (Johnson & Johnson) and Flexigel (Smith & Nephew). The same procedure described above was performed for these products, with data shown in FIG. 2. The XCell data used was for material containing 4.3% cellulose. As is clearly evident, the XCell dressing donated over 75% of its initial weight, outperforming all competitor products, which donated between 9% and 31%.

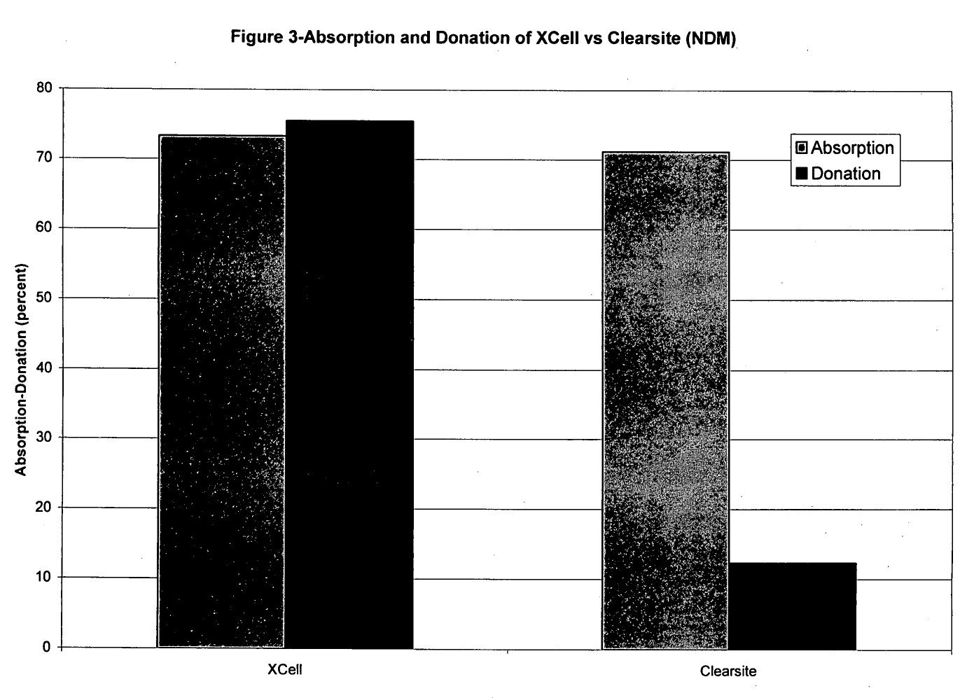

Although donation is very important for both wound healing and aesthetic epidermal/dermal improvement, a dressing would be ideal if it had the ability to donate and absorb. The procedure described previously for absorption was used to test Clearsite hydrogel wound dressing. The data for this is shown in FIG. 3, along with donation data and XCell data. As can be seen, the absorption of both samples is nearly identical, but the XCell material donated six times more moisture than the hydrogel.

### Example 2

### Biocompatibility Testing

The sterile cellulose dressing was subjected to the following biocompatibility tests: 1) Guinea pig sensitization, 2) Primary irritation in rabbits and, 3) Cellular cytotoxicity. In the sensitization test, extracts of the product were injected into six guinea pigs. The body temperatures of the guinea pigs were monitored for any sensitization reaction during the 8-10 week study period. The results showed no evidence of delayed dermal contact sensitization in the guinea pigs. The Primary irritation test was a two-week study using rabbits. In this test extracts of the cellulose dressing were injected subcutaneously and the skin was observed for any irritation reactions. The results showed that there was no evidence of significant irritation or toxicity from the subcutaneous injection of the extract into rabbits. The Primary Irritation Index of the cellulose dressing extract was found to be negligible. Finally, the cytotoxicity of the dressing with mammalian cells was tested using murine L929 cell culture. The results indicated that an extract of the cellulose dressing was not cytotoxic and did not inhibit cell growth. The cellulose dressing of the instant invention successfully passed all of these tests thus assuring that the product is biocompatible and safe

### Example 3

### Wound Healing in Animal Models

The objective of animal pre-clinical studies was to compare the wound healing performance in animal porcine models of the microbial derived cellulose wound dressing with existing wound dressing products such as hydrocolloids and hydrogels.

The test was conducted using the porcine model protocol of the Department of Dermatology of the University of Miami School of Medicine in compliance with Association for Accreditation of Laboratory Animal Care (AAALAC).

Briefly, the test was conducted on 2 pathogen-free pigs over a seven-day period. Approximately 140 rectangular wounds (10×7×0.3 mm) were made in the paravertebral and thoracic area of each pig with a specialized electrokeratome fitted with a 7 mm blade. The wounds are separated from one another by a 15 mm of unwounded skin. About 35 wounds were randomly assigned to each wound dressing treatment group of cellulose, hydrocolloid, hydrogel and no dressing/air exposed. An epidermal migration assessment was started two days after application.

In summary, the results showed that the cellulose wound dressing healed the partial thickness wounds as well as the hydrocolloid dressing and better than the hydrogel dressing. Significantly, on the fourth day after wounding, the cellulose wound dressing healed 70% of the wounds as compared to 50%, 20% and 0% for the hydrocolloid, hydrogel and air-exposed wounds, respectively. By the fifth day, both cellulose and hydrocolloid dressings had both healed 100% of the sampled wounds, while the hydrogel and air exposed samples were only 70% and 50% healed, respectively.

### Example 4

### Testing of Cellulose Dressing with Telfa

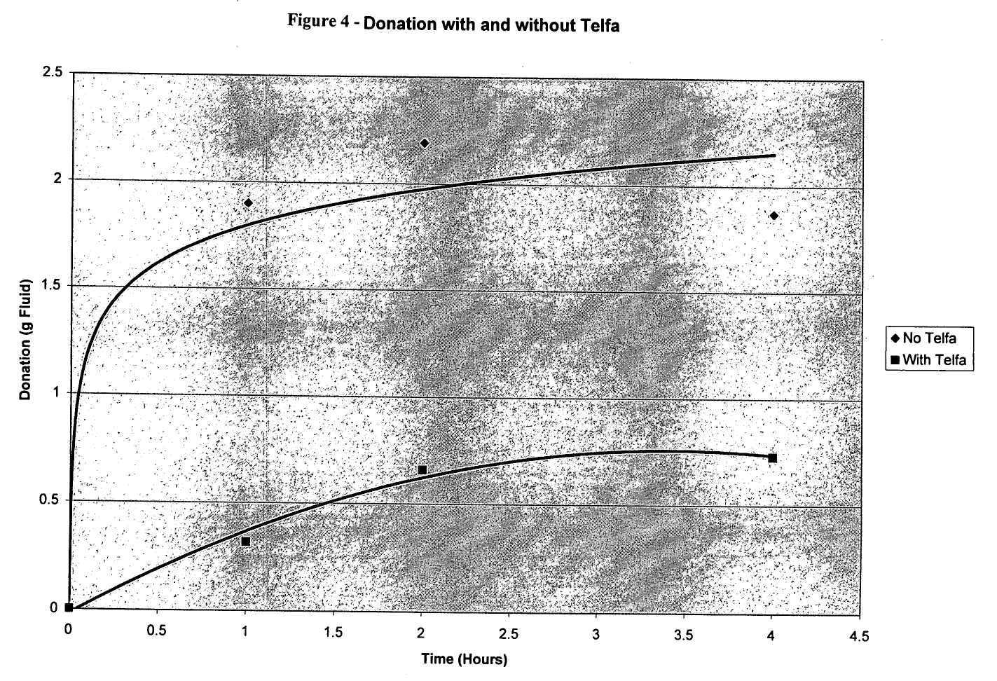

Donation testing per example 1 was also performed on samples where a sheet of Telfa® was placed between the leather and XCell Dressing. Values of grams of fluid donated for materials with and without Telfa were plotted at 0, 1, 2 and 4 hours. FIG. 4 illustrates the results and demonstrates the decreased donation over time when Telfa is placed between the leather and XCell.

### Example 5

### Human Clinical Effectiveness

Based on the examples disclosed in this specification and a previously performed human clinical evaluation of XCell on venous stasis ulcers, the function of the cellulose wound dressing applied as a human facial mask can be determined. The clinical study was a multi-center randomized controlled evaluation on 49 patients. Wounds were dressed with either XCell or standard of care and compression. Weekly dressing changes were performed for 12 weeks or until wound healing. Results from that study suggest that the cellulose face mask would likewise be expected to reduce pain and discomfort of the wound. The dressing would also exhibit strength in the removal of slough necrosis and maintenance of a moist environment for faster epithelial migration. Additionally, treatment with the cellulose dressing would result in a better aesthetic outcome and faster healing time. Other notable benefits of the use of the cellulose dressing on a human patient would include 1) reducing erythema—(inflammation) both in severity and duration, 2) preventing the formation of crust that may impede epithelialization, 3) reducing edema or swelling, Also, the cellulose dressing would prevent the occurrence of pruritus (itching) and purpura (small lesions) as well as lower the incidence of hyperpigmentation and hyperpigrnentation. Additionally, the dressing could be used for depigmentation and whitening in human patients.

The dressing would also prevent any infection or contact dermatitis in human patients. Ease of dressing removal after use and reduced/flexible dressing changes leading to less disturbance of the wound bed and/or treatment area are additional desirable features. Overall, the use of cellulose dressing promoted proper skin maturation and remodeling thereby producing an improved the quality of skin-tightness and roughness.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

1. A method for treating epidermal/dermal tissue comprising:
(a) applying a nonpyrogenic, biocompatible microbial-derived cellulose dressing to the desired epidermal/dermal tissue site; and changing the dressing from twice daily to weekly, and
(b) wherein said microbial-derived cellulose dressing comprises from about 1.5% to about 9% cellulose by weight.

2. The method of claim 1, wherein the microbial-derived cellulose dressing is comprised of about 3% to about 7% cellulose by weight.

3. The method of claim 1, wherein the microbial-derived cellulose dressing is comprised of about 4% to about 6% cellulose by weight.

4. The method of claim 1, wherein the cellulose dressing is used for aesthetic application.

5. The method of claim 4 wherein the aesthetic application is selected from the group comprising a chemical peel, dermabrasion, laser resurfacing, and other skin treatments.

6. The microbial-derived cellulose dressing of claim 1 wherein the dressing comprises the shape of the application area.

7. The dressing of claim 6 wherein the shape is selected from the group comprising a circle, oval, square, rectangle, triangle, or any other geometric shape.

8. The dressing of claim 6 wherein the shape is cut to fit a specific treatment area.

9. The dressing of claim 6 wherein the shape comprises half a face.

10. The dressing of claim 9 wherein the shape comprises the right or left side of the face.

11. The dressing of claim 9 wherein the shape comprises the top half of the face comprising the nose area up to the forehead.

12. The dressing of claim 11 wherein the shape comprises the dimensions of about 8″ to 12″ width by 4″ to 7″ height.

13. The dressing of claim 11 wherein the shape comprises openings for eyeholes.

14. The dressing of claim 9 wherein the shape comprises the lower half of the face comprising below the nose to the bottom of the chin or jaw.

15. The dressing of claim 14 wherein the shape comprises an opening for the mouth.

16. The dressing of claim 14 wherein the shape comprises the dimensions of about 8″ to 12″ width by 2″ to 6″ height.

17. The method of claim 1 wherein biocompatibility is determined by
(a) a passing response to a primary irritation test in rabbits,
(b) a passing response to a cytotoxicity test using murine L929 cells, and
(c) a passing response to a guinea pig sensitization test

18. The method of claim 1 wherein the microbial-derived cellulose dressing donates up to about 100% of its liquid weight and absorbs up to about 200% of its weight.

19. The microbial-derived cellulose dressing of claim 18 comprising about 3 to about 7 wt. % of cellulose.

20. The microbial-derived cellulose dressing of claim 18 comprising about 4 to about 6 wt. % of cellulose.

21. The method of claim 18, wherein the cellulose dressing is used for aesthetic application.

22. The method of claim 18 wherein the aesthetic application is selected from the group comprising a chemical peel, dermabrasion, laser resurfacing and other skin treatments.

23. The microbial-derived cellulose dressing of claim 18 wherein the dressing comprises the shape of the application area.

24. The dressing of claim 23 wherein the shape is selected from the group comprising a circle, oval, square, rectangle, triangle, or any other geometric shape.

25. The dressing of claim 23 wherein the shape is cut to fit a specific treatment area.

26. The dressing of claim 23 wherein the shape comprises half a face.

27. The dressing of claim 26 wherein the shape comprises the right or left side of the face.

28. The dressing of claim 26 wherein the shape comprises the top half of the face comprising the nose area up to the forehead.

29. The dressing of claim 28 wherein the shape comprises the dimensions of about 8″ to 12″ width by 4″ to 7″ height.

30. The dressing of claim 28 wherein the shape comprises openings for eyeholes.

31. The dressing of claim 26 wherein the shape comprises the lower half of the face comprising below the nose to the bottom of the chin or jaw.

32. The dressing of claim 31 wherein the shape comprises an opening for the mouth.

33. The dressing of claim 31 wherein the shape comprises the dimensions of about 8″ to 12″ width by 2″ to 6″ height.

34. The microbial-derived cellulose dressing of claim 18 wherein biocompatibility is determined by
(a) a passing response to a primary irritation test in rabbits,
(b) a passing response to a cytotoxicity test using murine L929 cells, and
(c) a passing response to a guinea pig sensitization test

35. A method for preparing a microbial-derived cellulose dressing comprising:
(a) statically producing a microbial cellulose pellicle using Acetobacter xylinum;
(b) isolating the pellicle with a cellulose to water ratio in the range of about 1:50 to about 1:500;
(c) incorporating additives into or onto the cellulose dressing and
(d) dehydrating the isolated pellicle to a cellulose content of 1.5 to 9 wt. %.

36. The microbial-derived cellulose dressing of claim 35 wherein the additive is selected from the group consisting of ascorbic acid; retinol; alpha hydroxy acid, alpha-tocopherol; vitamins A, C, E and/or their salts and esters; or any combination thereof.

37. The microbial-derived cellulose dressing of claim 35 wherein the ascorbic acid and or it's salts comprises about 0.5 to 10 wt. %]

38. The microbial-derived cellulose dressing of claim 37 wherein the percentage of ascorbic acid is about 1 wt. %.

39. The microbial-derived cellulose dressing of claim 36 wherein Vitamin E and/or it esters comprises about 0.1 to 10 wt. %

40. The microbial-derived cellulose dressing of claim 39 wherein the percentage of Vitamin E is about 0.5 wt. %.

41. The microbial-derived cellulose dressing of claim 36 wherein Alpha-OH-acid-Glycolic acid comprises about 2.0 to 10 wt. %.

42. The microbial-derived cellulose dressing of claim 41 wherein the percentage of Alpha-OH-acid-Glycolic acid is about 4.0 wt. %.

43. The microbial-derived cellulose dressing of claim 36 wherein Retinoic Acid or its variants comprises about 0.01 to 0.25 wt. %

44. The microbial-derived cellulose dressing of claim 43 wherein the percentage of Retinoic Acid is about 0.1 wt %.

45. The microbial-derived cellulose dressing of claim 36 wherein Retinol comprises about 0.05 to 2.0 wt. %

46. The microbial-derived cellulose dressing of claim 45 wherein the percentage of Retinol is about 0.1 wt. %.

47. The microbial-derived cellulose dressing of claim 35 where the additive is a medicament selected from the group consisting of a MMP inhibitor, growth factor, epithelial growth factor (EGF), Bone Morphogenetic Protein (BMP), PHMB (polyhexamethylene biguanide), or a combination thereof.

48. The microbial-derived cellulose dressing of claim 35 where the additive is a fragrance.

49. A kit comprising:
(a) a microbial-derived cellulose comprising about 1.5 to about 9 wt. % of cellulose;
(b) a moisture proof package containing said microbial-derived cellulose; and
(c) instructions for applying the microbial-derived dressing.

50. The kit of claim 49, wherein the microbial-derived cellulose comprises about 3 to about 7 % cellulose.

51. The kit of claim 49, wherein the microbial-derived cellulose comprises about 4% to about 6 % cellulose.

52. The kit of claim 49 wherein the microbial-derived cellulose comprises the addition of medicaments chosen from the group consisting of ascorbic acid, retinol, alpha-tocopherol, vitamins A, C, E, MMP inhibitor, growth factor, epidermal growth factor (EGF), Bone Morphogenetic Protein (BMP), PHMB (polyhexamethylene biguanide),or any combination thereof.

53. The kit of claim 49 wherein the microbial-derived cellulose comprises the addition of a fragrance.

54. The kit of claim 49 which is sterilized by gamma irradiation.

55. The kit of claim 49, wherein the moisture-proof package containing the microbial-derived cellulose comprises an aluminum plastic-coated heat sealable chevron pouch.
